SEQUENCE LISTING (8 sequences)

SEQ ID NO: 1 - DNA, 12 bp, Neisseria gonorrhoeae
  source 1..12: mol_type = genomic DNA
atgccgtctgaa

SEQ ID NO: 2 - DNA, 32 bp, synthetic construct
  source 1..32: mol_type = other DNA
aaaaaaaaaatgaccatgccgtctgaacaaac

SEQ ID NO: 3 - DNA, 22 bp, synthetic construct
  source 1..22: mol_type = other DNA
gtggtttcagacggcatttgtg

SEQ ID NO: 4 - DNA, 12 bp, synthetic construct
  source 1..12: mol_type = other DNA
ttcactgatgtg

SEQ ID NO: 5 - DNA, 12 bp, synthetic construct
  source 1..12: mol_type = other DNA
ttcagacgtgtg

SEQ ID NO: 6 - DNA, 12 bp, synthetic construct
  source 1..12: mol_type = other DNA
ttcagacgtgtg

SEQ ID NO: 7 - DNA, 12 bp, synthetic construct
  source 1..12: mol_type = other DNA
ttcagacggctg

SEQ ID NO: 8 - DNA, 12 bp, synthetic construct
  source 1..12: mol_type = other DNA
ttcagacggcat

(19) United States
(12) Patent Application Publication
(10) Pub. No.: US 2025/0305069 A1
(43) Pub. Date: Oct. 2, 2025

(54) DIAGNOSTIC METHOD AND KIT

(71) Applicant: THE UNIVERSITY OF BRISTOL, Bristol (GB)

(72) Inventors: Darryl HILL, Bristol (GB); Sean DAVIS, Bristol (GB); Ella CARTER, Bristol (GB)

(73) Assignee: THE UNIVERSITY OF BRISTOL, Bristol (GB)

(21) Appl. No.: 18/863,095

(22) PCT Filed: May 4, 2023
(86) PCT No.: PCT/EP2023/061802

§ 371 Date: Nov. 5, 2024

(30) Foreign Application Priority Data
May 6, 2022 (GB) 2206645.0

(51) Int. Cl. C12Q 1/689; C12Q 1/6834

(52) U.S. Cl. CPC C12Q 1/689; C12Q 1/6834

(57) ABSTRACT

Detecting target genomic DNA in a test sample, wherein the target genomic DNA comprises a DNA repeat sequence, using a nanoprobe comprising a nanoparticle functionalised by a surface-mounted oligonucleotide, wherein the oligonucleotide comprises a sequence that is substantially complementary to a portion of the DNA repeat sequence, and a nanoprobe and kit related thereto.

### TECHNICAL FIELD

This invention relates to the detection of target genomic DNA that comprises a DNA repeat sequence, using a nanoprobe. In particular, the nanoprobe comprises a nanoparticle functionalised by a surface-mounted oligonucleotide, wherein the oligonucleotide comprises a sequence that is substantially complementary to a portion of the DNA repeat sequence.

### BACKGROUND

The rapid spread of drug resistant pathogens, such as the Gram-negative bacteria Neisseria gonorrhoeae, continues to pose a serious threat to global health. To ensure timely treatment of infections, rapid diagnosis is critical. Nucleic acid amplification tests (NAATs) offer good sensitivity and specificity but can be unsuitable in some resource poor or remote clinics due to the need for trained professionals and specialised laboratory equipment. In addition, results typically take upwards of a week to be returned to the patient, which is particularly problematic in low to middle income countries (LMIC's) where patient return rates are low. As such, many clinics in resource-poor settings rely on Gram-strain based identification which provides poorer sensitivity and specificity than NAAT's, often leading to a missed or misdiagnosis. Such methods require overnight cell culturing and so results still take upwards of a day to be returned to the patient.

Consequently, there is a considerable need for an accurate, rapid, affordable and robust point-of-care diagnostic for infections that can be applied in these settings.

Metallic nanoparticles (NPs), have been extensively explored as markers for a range of point-of-care diagnostics. Most notably, gold nanoparticles (AuNPs) have been widely employed in pregnancy tests and more recently, COVID-19 lateral flow diagnostics. In particular, AuNP aggregation results in a distinct colour change (from red in a dispersed phase to blue in an aggregated phase; attributed to changes in surface plasmon resonance). By exploiting these unique optical properties of AuNPs, colour-based diagnostics have been developed for the detection of target DNA sequences. In particular, gold nanoparticles can be readily functionalised by surface-mounting of oligonucleotides to form ‘gold nanoprobes’, typically through binding of the oligonucleotide by terminal thiol groups to the AuNPs (N. L. Rosi and C. A. Mirkin, Chem. Rev., 2005, 105, 1547-156; J. M. Carnerero et al, Chemphyschem, 2017, 18, 17-33). Due to the highly-specific nature of complimentary base pairing, the gold nanoprobes can be designed to bind selectively to complimentary target sequences present in single-stranded DNA or RNA.

An early example exploiting gold nanoprobes for detection of specific DNA sequences was achieved by Elghanian et al. in 1997 (R. Elghanian, et al., Science, 1997, 277, 1078-1081), where a mixture of two types of gold nanoprobe was used to detect a target DNA sequence.

Each of the two different nanoprobe types had an oligonucleotide that was complementary to different sections of the target DNA sequence. As such, when the two types of gold nanoprobe bound to a single target DNA molecule, this brought the two types of gold nanoprobe into close proximity. This constituted an aggregation event and the colour was seen to change from red to blue, confirming the presence of the target DNA. In this way, the target DNA molecule could be exploited to draw together different gold nanoprobe types and cause the colour change (i.e. a colour change indicates presence of the target DNA). A disadvantage of this assay is the requirement for multiple nanoprobe types.

An alternative assay setup allows for using a single nanoprobe type. This assay setup relies upon using a nanoprobe aggregation agent, such as a charge screening agent, to induce aggregation of nanoprobes. In this version of the assay, conjugation of the nanoprobe to the target DNA or RNA is used to protect the nanoprobes from aggregation (i.e. a resistance to colour change upon adding the aggregation agent indicates presence of the target DNA).

This type of assay has been utilised in the detection of pathogenic DNA for the diagnosis of infectious diseases by designing the oligonucleotide such that it can anneal to a section of DNA sequence that is specific to the target pathogen. An important consideration in using such assays in rapid diagnosis of pathogens is the sensitivity. A higher sensitivity allows for a positive result to be achieved with lower quantities of DNA. When used for detection of pathogens, this means positive results can be achieved at lower numbers of pathogenic genomic DNA molecules (i.e. lower numbers of pathogen cells or particles). Depending on the pathogen, this may help achieve detection at clinically relevant pathogen concentrations or may help achieve detection at earlier stages of infection, without needing to use cell culture or DNA amplification techniques.

Reported examples of this technique include the following. Tunakhun et al. reported detection of N. gonorrhoeae by configuring a AuNP oligonucleotide to target the PorB gene, and reported a detection limit of 20 ng/μl N. gonorrhoeae DNA (P. Tunakhun et al., Biomed. Res., 2019, 30:2, 371-375). Liandris et al. reported detection of Mycobacterium by configuring a AuNP oligonucleotide to target a conserved genomic region within the 16s-23s internal transcribed spacer gene, and reported a detection limit of 18.75 ng diluted in 10 μl (E. Liandris et al., J. Microbiol. Methods, 2009, 78, 260-264). Bakthavathsalam et al. have reported detection of E. coli by configuring a AuNP oligonucleotide to target the malB gene (P. Bakthavathsalam et al., J. Nanobiotechnology, 2012, 10, 8). Andreadou et al. have reported detection of the protozoa Leishmania by targeting AuNPs to four separate regions of the kinetoplast minicircle DNA. Leishmania comprises high copy numbers of the kinetoplast DNA molecules per cell (i.e., providing for a greater number of short DNA molecules than the single long molecule of genomic DNA found per cell). A sensitivity of 11.5 ng/μl Leishmania DNA was reported.

The present invention aims to improve one or more aspects of detecting target DNA using nanoprobes.

### SUMMARY OF THE INVENTION

According to a first aspect, the invention provides a method for detecting target genomic DNA in a test sample, wherein the target genomic DNA comprises a DNA repeat sequence, said method comprising: (a) providing a nanoprobe comprising a nanoparticle functionalised by a surface-mounted oligonucleotide, wherein the oligonucleotide comprises a sequence that is substantially complementary to a portion of the DNA repeat sequence, (b) contacting the test sample with the nanoprobe, (c) contacting the test sample with a nanoprobe aggregation agent, and (d) assessing the amount of nanoprobe aggregation.

When target DNA is present, nanoprobe aggregation is reduced compared with when there is no target DNA in a similar test sample.

In one embodiment, the target genomic DNA is genomic DNA of a target pathogen. In this embodiment, wherein the target pathogen can be a prokaryote, preferably a bacterium, more preferably a Gram-negative bacterium, yet more preferably Neisseria gonorrhoeae. Where the target genomic DNA is genomic DNA from a pathogen, presence of the DNA is indicative of presence of the pathogen in the sample.

In an embodiment, the DNA repeat sequence can comprise at least 2, 4, 6, 8, 10, 20, 40, 60, 80, 100, 200, 400, 600, 800, 1000 or 1500 repeats (i.e. DNA repeat units). It is particularly preferred that the DNA repeat sequence has at least 100 DNA repeat units.

In an embodiment, the DNA repeat sequence can be a DNA uptake sequence, preferably a DNA uptake sequence in the genomic DNA of a bacterial species, particularly within the Neisseriaceae family or Pasteurellaceae family, more preferably a DNA uptake sequence in the genomic DNA of Neisseria gonorrhoeae or Haemophilus influenzae.

In an embodiment, the oligonucleotide comprises a sequence that is complementary to a portion of the DNA repeat sequence. In one embodiment, the oligonucleotide comprises a sequence having at least 6 contiguous nucleotides that are complementary to a portion of the DNA repeat sequence, i.e., to 6 contiguous nucleotides in the DNA repeat sequence.

In an embodiment, the nanoparticle exhibits a different colour in a disperse state versus an aggregated state.

In an embodiment, the DNA aggregation agent is a charge screening agent, preferably a salt, more preferably a cation. In a preferred embodiment, the nanoprobe aggregation agent is a magnesium salt. In this embodiment, it is preferred that the magnesium salt is added to the test sample in an amount that gives a magnesium salt concentration in the test sample of between 20 and 60 mM, preferably between 30 and 50 mM, more preferably about 40 mM.

In one embodiment, the target genomic DNA is the DNA uptake sequence of Neisseria gonorrhoeae, and the nanoprobe comprises a gold nanoparticle functionalised by an oligonucleotide comprising a sequence that is complementary to at least 8 contiguous nucleotides of the DNA uptake sequence.

According to a second aspect, the invention provides a method for detecting the presence of a target pathogen in a test sample, wherein the target pathogen comprises genomic DNA that comprises a DNA repeat sequence, said method comprising: (a) providing a nanoprobe comprising a nanoparticle functionalised by a surface-mounted oligonucleotide, wherein the oligonucleotide comprises a sequence that is substantially complementary to a portion of the DNA repeat sequence, (b) contacting the test sample with the nanoprobe, (c) contacting the test sample with a nanoprobe aggregation agent, and (d) assessing the amount of nanoprobe aggregation.

In a preferred embodiment, the test sample is a sample obtained from a subject, and detection of a target pathogen is indicative of infection by the target pathogen.

According to a third aspect, the invention provides a method of diagnosing infection with a target pathogen in a subject, using a test sample obtained from the subject, wherein the target pathogen comprises genomic DNA that comprises a DNA repeat sequence, said method comprising: (a) providing a nanoprobe comprising a nanoparticle functionalised by a surface-mounted oligonucleotide, wherein the oligonucleotide comprises a sequence that is substantially complementary to a portion of the DNA repeat sequence, (b) contacting the test sample with the nanoprobe, (c) contacting the test sample with a nanoprobe aggregation agent, and (d) assessing the amount of nanoprobe aggregation.

According to a fourth aspect, the invention provides a nanoprobe comprising a nanoparticle functionalised by a surface-mounted oligonucleotide, wherein said oligonucleotide comprises a sequence that is substantially complementary to a portion of a DNA repeat sequence of target genomic DNA, wherein the target genomic DNA is genomic DNA of a target pathogen.

According to a fifth aspect, the invention provides a nanoprobe according to the fourth aspect, for use in the diagnosis of infection by the target pathogen.

According to a sixth aspect, the invention provides a kit comprising: (a) an analytical sample comprising a nanoprobe according to the fourth aspect, and (b) a nanoprobe aggregation agent.

According to a seventh aspect, the invention provides a method for designing a nanoprobe, the method comprising: (a) identifying a target pathogen comprising genomic DNA comprising a DNA repeat sequence, (b) identifying the sequence of the DNA repeat sequence, (c) designing an oligonucleotide that is substantially complementary to a portion of the DNA repeat sequence, and (d) generating the nanoprobe by surface-mounting the oligonucleotide on a nanoparticle.

The inventors have provided an improved method of detecting genomic DNA comprising a repeat sequence. In one embodiment, the targeting of DNA repeat sequences by the nanoprobes was seen to improve the sensitivity over previous methods, where those previous methods utilised targeting of a sequence found only once on a DNA molecule. Despite DNA repeat sequences having the potential capacity to bind multiple nanoprobes in close proximity and thereby cause aggregation, targeting of DNA repeat sequences was found by the inventors not to cause aggregation. Furthermore, targeting of DNA repeat sequences was found to provide a significant improvement to sensitivity. In one example, using gonococcal DNA as a target, a previously disclosed method that involved targeting a gonococcal gene (porB) gave a detection limit of 20 ng/μl (120 ng) genomic DNA (P. Tunakhun et al., Biomed. Res., 2019, 30:2, 371-375). In contrast, targeting of a gonococcal DNA repeat sequence (DNA Uptake Sequence; DUS) gave a detection limit of 2.5 ng/μl genomic DNA, which represents an 8-fold improvement in sensitivity. Without wishing to be bound by theory, it is thought that the enhanced sensitivity may ultimately derive from multiple nanoprobe-target annealing events, potentially conferring additional stability to the nanoprobe due to additional steric effects and/or electronic stabilisation.

This distinct improvement in sensitivity clearly demonstrates the advantage of targeting a repeat DNA sequence. In this example, the detection limit is equivalent to ˜6.2 million gonococcal cells, which is of a similar order of magnitude to the average bacterial load present in patient urethal swabs (D. Priest et al, Sex. Transm. Infect., 2017, 93, 478-481). Therefore, the sensitivity of this diagnostic may omit the need for highly demanding and timely cell culture or DNA-amplification methods, as required for NAATs. Moreover, a positive result can be obtained within 30 minutes of application to the test sample, compared to NAATs which take between 1-3 days for results to be returned to the patient. Yet furthermore, this result can be obtained without complex equipment, procedures or knowledge being required. Thus, this novel approach allows for a distinct speed and affordability advantage over NAATs which could allow for use in resource-poor clinics and point-of-care applications.

Thus, in some embodiments, the method does not comprise a cell culture step, does not comprise a DNA amplification step and/or does not comprise a DNA digestion step.

### DNA Repeat Sequences

The invention relates to detection of target genomic DNA that comprises a DNA repeat sequence. By “DNA repeat sequence” we are referring to a sequence that is found more than once in the genomic DNA. The specific string of nucleotides that is repeated can be referred to as a DNA repeat unit. In an embodiment, the DNA repeat unit can be defined as the shortest sequence of non-repeating nucleotides. In other words, the DNA repeat unit itself does not comprise any DNA repeats. The DNA repeat sequence can comprise a plurality of DNA repeat units. Where applicable, reference in this specification to properties of a DNA repeat sequence can also apply to properties of its DNA repeat unit.

Preferably, the DNA repeat unit comprises at least 4, 5, 6, 7, 8, 9 or 10 contiguous nucleotides. It is particularly preferred that the DNA repeat unit comprises at least 8 contiguous nucleotides. The DNA repeat unit does not have a particular upper limit for the number of contiguous nucleotides. In certain embodiments, the DNA repeat unit can have fewer than 100, 90, 80, 70, 60, 50, 40, 30, 20, 19, 18, 17, 16, 15, 14, 13, 12 or 11 contiguous nucleotides.

In one embodiment, the DNA repeat sequence comprises a plurality of DNA repeat units that are contiguous in the genomic DNA. In other words, at least one DNA repeat unit starts just after another repeat unit. In this embodiment, there may be more than one plurality of DNA repeat units that are contiguous in the genomic DNA. In another embodiment, all of the repeat units in the genomic DNA are contiguous.

In another embodiment, the DNA repeat sequence comprises DNA repeat units that are dispersed throughout the genomic DNA. In other words, a DNA repeat unit does not have another DNA repeat unit contiguous to it in the 3′ or 5′ direction. In one embodiment, no two DNA repeat units are contiguous in the genomic DNA. In this embodiment, the DNA repeat units are separated by at least 1, 2, 6, 8, 10, 20, 40, 60, 80, 100, 200, 400, 600, 800, 1000 or 1500 nucleotides. In one embodiment, the DNA repeat sequence can comprise at least one plurality of DNA repeat units that are contiguous in the genomic DNA and at least one DNA repeat unit that is dispersed throughout the genomic DNA.

In one embodiment, the DNA repeat sequence comprises at least 4, 6, 8, 10, 20, 40, 60, 80, 100, 200, 400, 600, 800, 1000, 1500, 2000, 2500, 5000, 10000, 20000, or 50000 DNA repeat units.

### Target Pathogens Having DNA Repeat Sequences

The target genomic DNA is preferably genomic DNA of a target pathogen. According to this embodiment of the invention, nanoprobe-based methods for detecting pathogens can be enhanced. In one embodiment, this embodiment can find utility in enhancing the sensitivity of such methods of detection.

The target pathogen is not particularly limited, except that it should have genomic DNA comprising a DNA repeat sequence. In a preferred embodiment, the pathogen is a prokaryote, preferably a bacteria, more preferably a Gram-negative bacteria, yet more preferably Neisseria gonorrhoeae.

In one embodiment, the DNA repeat sequence is a DNA-Uptake Sequence (DUS). In this embodiment, the DUS can be the DUS of a bacteria, such as a Gram-negative bacteria. The DUS can a DUS in the genomic DNA of a bacterial species within the Neisseriaceae family or Pasteurellaceae family. In one embodiment, the DUS is of Neisseria gonorrhoeae or Haemophilus influenzae. In a particularly preferred embodiment, the DNA repeat sequence is the DUS of Neisseria gonorrhoeae.

Depending on the target pathogen, the DNA repeat sequence can be specific to a species or may be able to detect multiple different pathogens within a family. For instance, there may be subtle dialect differences in the DUS sequences within species in the families Neisseriaceae or Pasteurellaceae (Bakkali 2021 Genomics 113 (2800-2811)). Those skilled in the art can use routine knowledge and procedures in the art of oligonucleotide complementarity to identify oligonucleotides specific to a species or general to different species within a family, depending on the specific genomic DNA sequences involved.

In one embodiment, the DNA repeat sequence is the Haemophilus influenzae DUS, which has 1465 repeats of a 9 bp DNA repeat unit AAGTGCGGT (see, for instance, WO2000015265A1).

In a particularly preferred embodiment, the DNA repeat sequence is the Neisseria gonorrhoeae DUS sequence, which has a 12-nucleotide repeating DNA repeat unit (5′-ATGCCGTCTGAA-3′) that can be present up to 2000 times in a single genome (S. A. Frye, et al., PLOS Genet., 2013, 9, e1003458). This is particularly advantageous because this specific DUS sequence is only found in high frequency within the genomes of Neisseria species.

### Oligonucleotide Sequence

The invention relates to an oligonucleotide comprising a sequence that is substantially complementary to a portion of the DNA repeat sequence. By ‘substantially complementary’, we mean that the oligonucleotide has sufficient complementary to the portion of the DNA repeat sequence to enable annealing of the oligonucleotide with the DNA repeat sequence. It is well known in the field that annealing does not always require full complementarity, and the skilled person can readily design and test sequences to identify oligonucleotides that depart from full complementarity yet retain the ability to anneal to the target DNA repeat sequence.

In one embodiment, the oligonucleotide comprises a sequence that is substantially complementary to a portion of the DNA repeat unit or is substantially complementary to the full length of the DNA repeat unit.

In a preferred embodiment, the oligonucleotide comprises a sequence that is fully complementary to a portion of the DNA repeat sequence. In a preferred embodiment, the oligonucleotide comprises a sequence that is complementary to a contiguous portion of the DNA repeat sequence.

In one embodiment, the oligonucleotide consists of at least 6, 7, 8, 9, 10, 11, 12, 13, 14, 15, 16, 17, 18, 19 or 20 nucleotides. In one embodiment, the oligonucleotide consists of less than 14, 15, 16, 17, 18, 19, 20, 25, 30, 40, 50, 60, 80 or 100 nucleotides. In a preferred embodiment the oligonucleotide consists of less than 19 nucleotides and in a particularly preferred embodiment the oligonucleotide consists of less than 15 nucleotides. In one embodiment, the oligonucleotide consists of 8 to 14 nucleotides. An oligonucleotide consisting of 10, 11 or 12 nucleotides was found to be particularly effective.

In one embodiment, the oligonucleotide comprises at least 6, 7, 8, 9, 10, 11, 12, 13, 14, 15, 16, 17, 18, 19 or 20 nucleotides that are complementary to a portion of the DNA repeat sequence or DNA repeat unit. In one embodiment, the oligonucleotide comprises up to 14, 15, 16, 17, 18, 19, 20, 25, 30, 40, 50, 60, 80 or 100 nucleotides that are complementary to a portion of the DNA repeat sequence or DNA repeat unit. In a preferred embodiment, all complementary nucleotides in the oligonucleotide are contiguous. In a particularly preferred embodiment, the oligonucleotide comprises at least 8 nucleotides that are complementary to a portion of the DNA repeat sequence. In a yet more preferred embodiment, the oligonucleotide comprises at least 8 contiguous nucleotides that are complementary to a contiguous stretch of 8 nucleotides in the portion of the DNA repeat sequence.

The sequence of the oligonucleotide that is substantially complementary to a portion of the DNA repeat sequence does not necessarily have to be the same length as the DNA repeat unit of the DNA repeat sequence. The sequence of the oligonucleotide that is substantially complementary to a portion of the DNA repeat sequence may be shorter than the DNA repeat unit. Alternatively, where the DNA repeat sequence comprises DNA repeat units that are contiguous on the genomic DNA, the oligonucleotide can be complementary to a sequence that spans more than one contiguous DNA repeat unit.

### Nanoprobe

The oligonucleotide is surface-mounted to a nanoparticle. The nanoparticle material is not particularly limited and can be selected from nanoparticle materials known in the field. In one embodiment, the nanoparticle is a metallic nanoparticle. In this embodiment, the metal may be chosen from metals known in the field to form nanoparticles. Suitable examples include gold, silver, copper, and related alloy and composite nanoparticles (e.g., nanoparticles comprising suitable core and shell materials). In a particularly preferred example, the nanoparticle is a gold nanoparticle (AuNP).

By nanoparticle, we are referring to a particle that has its largest dimension in the nanoscale range. In one embodiment, the largest dimension of the nanoparticle is between 1 nm and 500 nm. Preferably the largest dimension is less than 500 nm, 400 nm, 300 nm, 200 nm, 100 nm, 90 nm, 80 nm, 70 nm, 60 nm, 50 nm, 40 nm, 30 nm or 20 nm. It is particularly preferred that the largest dimension is less than 40 nm. Preferably the largest dimension is greater than 1 nm, 2 nm, 3 nm, 4 nm, 5 nm, 6 nm, 7 nm, 8 nm, 9 nm, or 10 nm. It is preferred that the largest dimension of the nanoparticle is between 1 nm and 100 nm, preferably between 5 nm and 40 nm, more preferably between 10 nm and 20 nm.

In one embodiment, the nanoparticle is a substantially spherical nanoparticle or a spherical nanoparticle. In this embodiment, the largest dimension of the nanoparticle is its diameter. Preferably the diameter is less than 500 nm, 400 nm, 300 nm, 200 nm, 100 nm, 90 nm, 80 nm, 70 nm, 60 nm, 50 nm, 40 nm, 30 nm or 20 nm. It is particularly preferred that the diameter is less than 40 nm. Preferably the diameter is at least 1 nm, 2 nm, 3 nm, 4 nm, 5 nm, 6 nm, 7 nm, 8 nm, 9 nm, or 10 nm. It is preferred that the nanoparticle has a diameter of between 1 nm and 100 nm, preferably between 5 nm and 40 nm, more preferably between 10 nm and 20 nm.

In a preferred embodiment, the nanoparticle is a substantially spherical gold nanoparticle having a diameter of between 1 nm and 100 nm, preferably between 5 nm and 40 nm, more preferably between 10 nm and 20 nm, preferably between 15 nm and 20 nm, yet more preferably about 18 nm.

Techniques for surface-mounting the oligonucleotide are known in the art. In one embodiment, the oligonucleotide comprises a surface-mounting functional group. In a preferred embodiment, the surface-mounting functional group is a thiol group. Preferably, the oligonucleotide is terminated by a surface-mounting functional group, such as a thiol group. In a particularly preferred embodiment, the oligonucleotide is a 5′-thiolated oligonucleotide. Techniques for introducing a thiol group at the 5′-end of oligonucleotides are known. Thiol groups are known to adhere to certain metals, such as gold, which allows for rapid and simple surface mounting of such oligonucleotides to gold nanoparticles.

In one embodiment, the oligonucleotide comprises a linker between the sequence that is substantially complementary to a portion of the DNA repeat sequence and the surface-mounting functional group. The linker can improve the ability of the sequence that is substantially complementary to a portion of the DNA repeat sequence to anneal to the DNA repeat sequence. The type of linker is not particularly limited and may be selected from linkers known in the field. The length of the linker is not particularly limited and may be selected and/or optimised by the skilled person using routine knowledge in the field.

In one embodiment, the linker does not comprise nucleotides. In one embodiment, the linker comprises nucleotides. In this embodiment, the linker can be at least 5, 6, 7, 8, 9, 10, 12, 14, 16, 18 or 20 nucleotides in length and/or up to 14, 16, 18, 20, 25, 30, 40, 50, 80 or 100 nucleotides in length. In one embodiment, the linker is between 5 and 80, preferably between 6 and 30, more preferably between 8 and 20, yet more preferably about 10 nucleotides in length. In one embodiment, the linker comprises a poly-adenosine nucleotide sequence. One advantage of having an all-nucleotide linker is that is facilitates straightforward synthesis and amplification using standard polynucleotide molecular biology techniques.

In another embodiment, the linker comprises polyethylene glycol (PEG). In one embodiment, the PEG comprises between 2 and 100, 3 and 50, 4 and 20, or 5 and 10 successive polyethylene glycol units. In a preferred embodiment, the PEG comprises 6 successive polyethylene glycol units.

Other techniques for surface-mounting the oligonucleotide to the nanoparticle can include techniques where the oligonucleotide is surface-mounted via non-nucleotide molecules. Suitable chemistries for achieving this, for example click-chemistry techniques, are known in the art.

In a particularly preferred embodiment, the oligonucleotide comprises a sequence that is complementary to a portion of the DNA repeat sequence, a polyadenosine linker, and a 5′-thiol functionality.

In one embodiment, the nanoparticle has an oligonucleotide loading density of at least 1, 2, 4, 6, 8, 10, 15 or 20 pmol cm⁻². In one embodiment, the nanoparticle has an oligonucleotide loading density of up to 100, 90, 80, 70, 60, 50, 40, or 30 pmol cm⁻². In one embodiment, the nanoparticle has an oligonucleotide loading density of 2 to 100 pmol cm⁻², preferably 10 to 50 pmol cm⁻², more preferably 15 to 30 pmol cm⁻². In a particularly preferred embodiment, the nanoparticle has an oligonucleotide loading density of up to 26 pmol cm⁻². In another embodiment, the nanoparticle is a gold nanoparticle having an 18 nm diameter, and an oligonucleotide loading density of up to 26 pmol cm⁻². The oligonucleotide loading density can be controlled using techniques known in the art. In one embodiment, oligonucleotide loading density can be controlled by design of the linker. For instance, polyethylene glycol (PEG) can be relatively bulky and negatively-charged, and can introduce greater steric and charge repulsion between DNA and nanoparticle, thereby limiting the formation of gold-thiol linkages.

Where the nanoparticle is a substantially spherical nanoparticle having a diameter of about 15 to 25 nm, the oligonucleotide loading density is preferably at least 50, 60, 80, 100, 120 or 140 oligonucleotides per nanoparticle and/or up to 500, 400, 300, 200, 180 or 150 oligonucleotides per nanoparticle. In one embodiment, the oligonucleotide loading density is 50 to 500, preferably 80 to 300, more preferably 120 to 180, yet more preferably about 145 oligonucleotides per nanoparticle.

### Test Sample

The method of detection is performed upon a test sample. The test sample may be any test sample where it is desirable to test for presence of a target genomic DNA that comprises a DNA repeat sequence. The test sample, by its nature, may or may not contain the target genomic DNA. The methods of the invention can be used to test for the presence or the absence of the target genomic DNA.

In a preferred embodiment, the method of detection is performed to detect the presence of a target pathogen in a test sample, wherein the target pathogen comprises genomic DNA that comprises a DNA repeat sequence.

The test sample, or source of the test sample, is not particularly limited. In one embodiment, the test sample may be from an environment suspected of containing a target pathogen, for example, a soil sample, food produce sample, or sample obtained from a human or animal subject. In a preferred embodiment, the test sample is obtained from a human or animal subject.

In a particularly preferred embodiment, the test sample is a bodily fluid. The bodily fluid is not particularly limited and may be a sample obtained from a body, such as a blood sample, urine sample, swab obtained from a body cavity, or a biopsy. Means for obtaining such samples are known in the art.

The methods of detection of the present invention can in some embodiments be used for methods of diagnosis. In one embodiment, this involves a method of diagnosing infection with a target pathogen in a subject, using a test sample obtained from the subject.

The method of detection involves contacting the test sample with the nanoprobe. By this, we mean that the test sample can be provided in a state to allow the nanoprobe to contact target genomic DNA, if present, under conditions that allow the nanoprobe to anneal with target genomic DNA. For instance, the test sample can be provided with single-stranded genomic DNA that will anneal with the nanoprobe without further manipulation. In some embodiments, however, further steps will be used to allow for any target genomic DNA to anneal with the nanoprobe. How this is achieved is not particularly limited, and will be understood by those skilled in the art of molecular biology.

For instance, the test sample may be provided in a state where the genomic DNA, if present, is in bulk solution (i.e., not contained within any cells or particles) and is denatured (i.e., single-stranded). Alternatively, the test sample may be provided in a state where the genomic DNA is contained within a cell and is not denatured.

The methods of the invention may comprise the step of subjecting the test sample to conditions suitable for releasing target genomic DNA from a cell or particle. Conditions for releasing genomic DNA from a cell or particle, such as from a target pathogen cell or target pathogen particle are known in the art and suitable conditions for any given target pathogen can be readily determined by the skilled person. For instance, conditions for releasing genomic DNA may include heating the test sample, adding a lysis solution, and/or exposing the test sample to ultrasonication, electroporation, and/or shear pressure.

The methods of the invention may comprise the step of subjecting the test sample to conditions suitable for denaturing genomic DNA. Conditions for denaturing genomic DNA are also known in the art. For instance, denaturing of genomic DNA is commonly conducted using heating.

Conditions for releasing genomic DNA from a cell and denaturing genomic DNA may be the same or different. Where they are different, they may be applied simultaneously, sequentially or separately. For instance, heat may be used to both release genomic DNA from a cell and denature the genomic DNA. Alternatively, release of the genomic DNA from a cell and denaturing of the genomic DNA can be conducted sequentially or separately, using different conditions. Typically, the genomic DNA will be released from a cell and then the genomic DNA will be denatured.

The nanoprobe may be contacted with the test sample prior to subjecting the test sample to conditions for releasing the genomic DNA and conditions for denaturing the genomic DNA. As such, the nanoprobe remains in the test sample through these steps. Alternatively, the nanoprobe may be contacted with the test sample after subjecting the test sample to conditions for releasing the genomic DNA and before conditions for denaturing the genomic DNA, or may be added after both of these steps.

The methods of the invention may further comprise the step of subjecting the test sample comprising the nanoprobe to conditions suitable for annealing the nanoprobe with the DNA repeat sequence. Conditions suitable for annealing of the nanoprobe oligonucleotide with the DNA repeat sequence of the genomic DNA are well-known in the art. In one embodiment, such conditions comprise cooling the test sample from a temperature at which the target genomic DNA would be denatured, to a temperature that allows for annealing of the nanoprobe oligonucleotide with the DNA repeat sequence of the genomic DNA. The test sample can be left for sufficient time for annealing of the nanoprobe with any target genomic DNA before moving to the next step.

In a preferred embodiment, the conditions suitable for denaturing the genomic DNA comprise heating the test sample comprising the nanoprobe to a temperature that is suitable for denaturing the genomic DNA and the conditions suitable for annealing of the nanoprobe oligonucleotide with the DNA repeat sequence comprise subsequently cooling the test sample to a temperature that allows for annealing of the nanoprobe oligonucleotide with the DNA repeat sequence of the genomic DNA.

The methods of the invention may further comprise subjecting the test sample to: (i) conditions suitable for releasing the genomic DNA from the target pathogen, (ii) subjecting the test sample to conditions suitable for denaturing the genomic DNA of the target pathogen, and (iii) subjecting the test sample comprising the nanoprobe to conditions suitable for annealing of the nanoprobe oligonucleotide with the DNA repeat sequence of the genomic DNA.

In general, the person skilled in the art will be familiar with techniques for releasing genomic DNA from a cell, denaturing genomic DNA, and annealing genomic DNA with oligonucleotides, as these are commonly used in other molecular biology techniques such as preparing samples for, and running, polymerase chain reaction amplification using oligonucleotide primers.

In one embodiment, the method for detection is conducted using a surface technique such as a lateral flow technique. In a preferred embodiment, the method for detection is conducted in solution, preferably aqueous solution. In other words, the step of contacting the test sample with the nanoprobe is conducted in solution, and remains in solution through the subsequent steps. In one embodiment, the nanoprobe is provided in solution, preferably aqueous solution. In one embodiment, the test sample is provided in solution, preferably aqueous solution. In one embodiment, both the nanoprobe and test sample are provided in solution, preferably aqueous solution. In another embodiment, the test sample may not be in solution, and is added to a nanoprobe that is provided in solution. For instance, this could involve taking a test sample such as a swab or a biopsy and adding to a nanoprobe that is provided in solution.

### Nanoprobe Aggregation Agent

The inventors have provided a nanoprobe that can bind to a portion of a DNA repeat sequence of genomic DNA. In so doing, the genomic DNA can provide a protective effect against nanoprobe aggregation agents. In an embodiment, the nanoprobe aggregation agent is an agent that can cause aggregation of nanoprobes that are not conjugated to genomic DNA and is an agent that does not cause aggregation of nanoprobes that are conjugated to the target genomic DNA. Such nanoprobe aggregation agents are known in the art.

In one embodiment, the nanoprobe aggregation agent is a salt. Utilising salt in this way is sometimes referred to as ‘salt challenge’.

In one embodiment, the nanoprobe aggregation agent is a charge screening agent. In one embodiment, the nanoprobe aggregation agent is a salt, preferably a cation. When a suitable salt is introduced to the system the Debye length decreases, screening the electrostatic charge. This can allow nanoparticles to approach each other such that strongly attractive Van Der Waals forces become dominant causing irreversible aggregation. The flocculation concentration for divalent counter-ions is on average about 100 times lower than for monovalent ions, and for trivalent ions about 1000 less.

In some embodiments, the nanoprobe aggregation agent may work over a preferred concentration range. The skilled person can readily screen concentrations to identify an effective concentration range.

In a preferred embodiment, the nanoprobe aggregation agent is a magnesium salt. In this embodiment, it is preferred that the magnesium salt is added to the test sample to give a final magnesium salt concentration of between 20 and 60 mM, preferably between 30 and 50 mM, more preferably about 40 mM.

After the nanoprobe aggregation agent has been added to the test sample, the amount of nanoprobe aggregation can be assessed. If the target genomic DNA is present in the test sample in a detectable amount, the amount of aggregation should be less than if the target genomic DNA is absent from the test sample. This can be readily determined by comparison of the amount of aggregation in the test sample versus a negative control sample that contains no target genomic DNA. A negative control sample may comprise no DNA. Alternatively, a negative control sample may comprise DNA that is not complementary to the surface mounted oligonucleotide. After the nanoprobe aggregation agent has been added to the test sample, the test sample can be provided sufficient time for aggregation of non-annealed nanoprobe to occur. The length of time that is sufficient can be readily determined through a negative control experiment where genomic DNA is absent.

The amount of nanoprobe aggregation may be assessed by means known in the art. For instance, light scattering can be used to assess the amount of nanoparticle aggregation.

In particular, it is preferred that the nanoparticle undergoes a colour change upon aggregation. It is noted that in this embodiment the nanoprobe does not require additional reporter molecules such as fluorophores or electrochemical reporter molecules. In an embodiment of this embodiment, the nanoprobe does not comprise a reporter molecule, such as a fluorophore or electrochemical reporter molecule. This can allow for straightforward assessment of the amount of nanoprobe aggregation by eye, without needing specialist equipment. In one embodiment, the nanoparticle comprises a metal that undergoes a colour change upon aggregation. This colour change may be caused by a change in surface plasmon resonance (SPR). Suitable metals that can undergo a colour change upon aggregation include gold, silver, and copper, and suitable alloys and composites thereof.

A colour change due to SPR can be dependent upon the nanoparticle size. The skilled person knows how to screen nanoparticle sizes using routine techniques in the art in order to identify suitable nanoparticle sizes.

In a preferred embodiment, the nanoparticle is a gold nanoparticle (AuNP). AuNPs are a popular choice for diagnostic applications because of their facile synthesis and bioconjugation, high stability and unique optical properties. In particular, AuNPs exhibit a characteristic surface plasmon resonance (SPR) which arises from collective oscillations of electrons in the conduction band of atomic gold. As a result, incident light which matches the frequency of this oscillation is strongly absorbed by the AuNPs. For spherical AuNPs below 100 nm, this absorption occurs in the visible region, and so the AuNPs exhibit a characteristic colour [X. Huang and M. A. El-Sayed, J. Adv. Res., 2010, 1, 13-28]. Aggregation of AuNP suspensions results in dipole-coupling between adjacent particles, causing a shift in the SPR to longer wavelengths (generally seen as a shift from red to blue). Hence, AuNP aggregation results in a distinct colour change that can be exploited for the colorimetric detection of aggregation.

### Kit

One aspect of the invention provides a kit comprising: (a) an analytical sample comprising a nanoprobe according to any preceding aspect of the invention, and (b) a nanoprobe aggregation agent. Depending on the ultimate use, the kit may additionally comprise suitable further components. For example, the kit may comprise a negative control sample comprising no target genomic DNA and/or a positive control sample comprising a DNA repeat sequence of the target genomic DNA. The kit can comprise further components to assist with release of the genomic DNA from a cell, such as a lysis buffer. The kit can comprise further components to assist with denaturation of the genomic DNA.

In one embodiment, the kit does not comprise cell culture components, does not comprise DNA amplification components and/or does not comprise DNA digestion components. As mentioned, in some embodiments, the methods of the invention can achieve high sensitivity without needing cell culture, DNA amplification or DNA digestion. This can also provide for a kit that has components that are stable without needing special handling, such maintaining the kit within a cold chain. Furthermore, this provides for a kit that can be used without needing complicated and numerous steps. This reduces points of failure and reduces the need for specialist training and/or equipment.

### Method of Designing a Nanoprobe

One aspect of the invention provides a method for designing a nanoprobe, the method comprising: (a) identifying a target pathogen comprising genomic DNA comprising a DNA repeat sequence, (b) identifying the sequence of the DNA repeat sequence, (c) designing an oligonucleotide that is substantially complementary to a portion of the DNA repeat sequence, and (d) generating the nanoprobe by surface-mounting the oligonucleotide on a nanoparticle.

Throughout the description and claims of this specification, the words “comprise” and “contain” and variations of the words, for example “comprising” and “comprises”, mean “including but not limited to”, and do not exclude other components, integers or steps. Moreover the singular encompasses the plural unless the context otherwise requires: in particular, where the indefinite article is used, the specification is to be understood as contemplating plurality as well as singularity, unless the context requires otherwise.

Preferred features of each aspect of the invention may be as described in connection with any of the other aspects. Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

### BRIEF DESCRIPTION OF THE DRAWINGS

One or more embodiments of the invention will now be described, by way of example only, with reference to the accompanying drawings, in which:

FIG. 1 shows characterisation of bare AuNPs and DNA-functionalised AuNPs (nanoprobes): a) TEM images of bare AuNPs: a)i-iv) individual images taken from random position on the TEM grid; b) Histogram of measured particle diameters for bare AuNPs; c)i-iii) TEM images of AuNP-DUS with uranyl acetate staining showing DNA conjugated to the particles surface; c)iv) TEM image of bare AuNP with same uranyl acetate staining—no peripheral staining was observed; d) UV-visible absorbance spectrum of bare AuNPs (black) and nanoprobes (grey) showing a 9 nm shift in surface plasmon resonance (SPR) upon DNA-functionalisation;

FIG. 2 shows a salt-induced aggregation test of gold nanoprobes treated with complementary or control oligonucleotides: a) photograph of nanoprobes after incubation with 50 μM of a reverse complement DUS (compDUS), heating to 95° C., before cooling to room temperature and treating with different concentrations of MgSO₄; b) photograph of nanoprobes after treatment as before but with 50 μM of random scramble oligonucleotide (SDU) in place of cDUS; c) UV-vis absorption spectra of nanoprobe samples treated with compDUS at increasing concentrations of MgSO₄—each experiment was repeated 3 times, with photographs shown from 1 repeat;

FIG. 3 shows salt-induced aggregation tests with partial complimentary oligonucleotides: a) photograph of gold nanoprobes after incubation with 50 μM of each oligonucleotide, heating to 95° C., before cooling to room temperature and treating with 40 mM MgSO₄; b) table of partial complimentary oligonucleotides used; c) UV-vis absorption spectra of nanoprobe samples with each oligonucleotide—each experiment was repeated 3 times, with photographs shown from 1 repeat;

FIG. 4 shows salt-induced aggregation tests with genomic DNA: a) photograph of nanoprobes after incubation with increasing concentrations of N. gonorrhoeae (MS11) genomic DNA, heating to 95° C., before cooling to room temperature and treated with 40 mM MgSO₄; b) photograph of nanoprobes after treatment as before but with E. coli (Stellar) genomic DNA; c) UV-vis absorption spectra of gold nanoprobe samples following treatment with increasing concentrations of N. gonorrhoeae DNA—each experiment was repeated 3 times, with photographs shown from 1 repeat.

FIG. 5 shows a scheme for gonococcal (Ng) DNA detection via salt-induced aggregation of gold nanoprobes: DUS-functionalised nanoprobes are incubated with a test sample of DNA. If the DNA contains the reverse complement DUS then the nanoprobes may anneal to the DNA, producing a double-stranded DNA corona around the nanoprobes. This improves both the steric and electronic stabilisation of the nanoprobes such that they remain stable at higher salt concentrations than nanoprobes which have not been treated with target DNA. E. coli (Ec) DNA is used as a control.

### DETAILED DESCRIPTION

### Materials & Methods

### Preparation and Characterisation of Gold Nanoparticles

Citrate-capped gold nanoparticles (AuNPs) were prepared using the Turkevich-Frens method (J. Kimling, M. Maier, B. Okenve, V. Kotaidis, H. Ballot and A. Plech, J. Phys. Chem. B, 2006, 110, 15700-15707). Gold (III) chloride trihydrate (10 mg, 0.025 mmol) was dissolved in 95 mL Milli-Q water and heated to a rolling boil with continuous stirring. 5 mL of 1% w/v sodium citrate dihydrate solution was added, and the mixture heated for 15 minutes, resulting is a colour change from pale yellow to a dark red solution. The AuNP solution was cooled and stored in the dark at 4° C. for several months. UV-visible spectroscopy (UV-vis) was used to confirm successful preparation and determine the AuNP molar extinction coefficient and solution concentration. The completed reaction mixture was deposited onto a 3.05 mm copper 400 mesh glow-discharged carbon-coated grid and imaged by a JEOL 1400 transmission electron microscope (TEM). Measurements were taken using ImageJ software to determine the average particle diameter from the collected TEM images. Dynamic light scattering (DLS) was used to measure the average hydrodynamic diameter and zeta potential.

### Preparation and Characterisation of DNA-Functionalised Gold Nanoprobes

A disulfide protected 5′-thiolated oligonucleotide (5′-[ThiC₆]aaaaaaaaaatgaccatgccgtctgaacaaac-3′, DUS sequence shown as bold text) was purchased from Sigma Aldrich. Prior to use, the oligonucleotide was deprotected by reduction with tris(2-carboxyethyl)phosphine (TCEP). For this, 30 μM oligonucleotide was added to 3 mM TCEP in 0.02 M HEPES buffer, pH 7.0. The oligonucleotide was reduced for 1 hour at room temperature, before purification using a Microspin G-25 column which has been prewashed with 0.01 M phosphate buffer, pH 7.5. The deprotected oligonucleotides were then conjugated to the bare AuNPs using a slow salt-aging process adapted from the method outlined Mirkin (S. J. Hurst, A. K. R. Lytton-Jean and C. A. Mirkin, Anal. Chem., 2006, 78, 8313-8318). Freshly cleaved oligonucleotide (4 nmol) was added to 1 mL of freshly synthesised AuNP suspension (˜1 nM) and the solution brought to 9 mM phosphate buffer, pH 7.5 and 0.1% SDS and shaken for 30 minutes. Over 48 hours, 6 incremental additions of 2 M NaCl were used to slowly bring the reaction to 0.3 M NaCl. The solution was then left shaking for a further 24 hours before centrifuging at 12,000 g for 25 minutes to pellet the gold nanoprobes. The nanoprobes were washed 3 times with 0.01 M phosphate buffer, 0.1 M NaCl, before redispersion in a storage buffer (1 mM phosphate buffer, 0.1 M NaCl and 0.1% SDS) at˜10 nM. The suspension was stored at 4° C. in the dark until further use.

The absorbance spectrum of the DNA-functionalised nanoprobe suspension was measured by UV-visible spectroscopy on a PerkinElmer Lambda 750. The average hydrodynamic particle diameter and zeta potential were measured by Dynamic Light Scattering (DLS) using a Malvern Zetersizer Nano. The nanoprobes were imaged using TEM. For this, dilute aqueous suspensions of the nanoprobes were deposited onto glow-discharged, carbon-coated 3.05 mm 400 mesh TEM grids and the excess liquid wicked away using filter paper. The TEM grids were then stained with 1% uranyl acetate for 30 seconds before washing three times with deionised water to create a positive stain. The grids were air dried overnight and imaged on a JEOL 1400 TEM.

To quantify the number of DNA strands attached to each particle, mercaptoethanol was used to cleave the gold-thiol bonds, thus detaching the DNA from the AuNP surface (M. S. Cordray, M. Amdahl and R. R. Richards-Kortum, Anal. Biochem., 2012, 431, 99-105). The solution was centrifuged at 14000 RPM and the supernatant combined with an Invitrogen™ Quant-iT™ OliGreen™ fluorescent reagent, designed specifically to bind and detect single-stranded oligonucleotides. Fluorescence emission was measured and compared to a calibration plot for different oligonucleotide concentrations, to determine the concentration of detached oligonucleotides.

### Salt-Induced Aggregation for the Detection of Complimentary Oligonucleotides

Gold nanoprobe suspension (3 μL) was added to an equal volume of a reverse complement DUS oligonucleotide (compDUS=5′-gtggtttcagacggcatttgtg-3′) to give a final oligonucleotide concentration of 50 μM. A non-complimentary oligonucleotide (SDU=5′-gtgattactgtccggaactgtg-3′) was used as a control. It is noted that the SDU control can be any non-complementary sequence. The suspensions were heated to 95° C. for 5 minutes and after cooling at room temperature for 15 minutes, MgSO₄ was added to give a final concentration between 0-100 mM. Each suspension was photographed, and particle aggregation observed visually. The UV-visible absorbance spectrum of each solution was collected between 400-700 nm using a PerkinElmer Lambda 750 UV-vis spectrophotometer. Each experiment was repeat 3 times.

### Testing the Sequence Specificity of Aggregation Tests

Gold nanoprobe suspension (3 μL) was added to an equal volume of partial matching complimentary oligonucleotides (sequences given in Table 1) to give final oligonucleotide concentrations of 50 μM. The mixtures were heated to 95° C. for 5 minutes and after cooling at room temperature for 15 minutes, MgSO₄ was added to give a final concentration of 40 mM. Particle aggregation was observed visually and by measuring the UV-vis absorbance of each solution between 400-700 nm. Each experiment was repeated 3 times.

TABLE 1
Table summarising the oligonucleotide sequences
used to test sequence specificity of the
nanoprobe aggregation-based diagnostic. In each
case the oligonucleotide was 12 bases long,
with increasing numbers of bases belonging
to compDUS shown in bold.

| Number of matching | |
|---|---|
| base pairs | Base sequence |
| 4 | 5′-ttcactgatgtg-3′ |
| 6 | 5′-ttcagacgtgtg -3′ |
| 8 | 5′-ttcagacgtgtg-3′ |
| 10 | 5′-ttcagacggctg-3′ |
| 12 | 5′-ttcagacggcat-3′ |

### Salt-Induced Aggregation for the Detection of Gonococcal Genomic DNA

Genomic DNA (gDNA) was extracted and purified from wild-type MS11 N. gonorrhoeae and Stellar Escherichia coli (E. coli) as a control, using a Qiagen DNeasy Blood and Tissue Kit. 3 μL of gold nanoprobe was added to an equal volume of N. gonorrhoeae or E. coli DNA to give a final concentration between 1-30 ng μL⁻¹. The mixture was heated to 95° C. for 5 minutes and after cooling at room temperature for 15 minutes, MgSO₄ was added to give a final concentration of 40 mM. Particle aggregation was observed visually and by measuring the UV-vis absorbance of each solution between 400-700 nm. Each experiment was repeat 3 times.

### Example 1—Preparation and Characterisation of Gold Nanoprobes

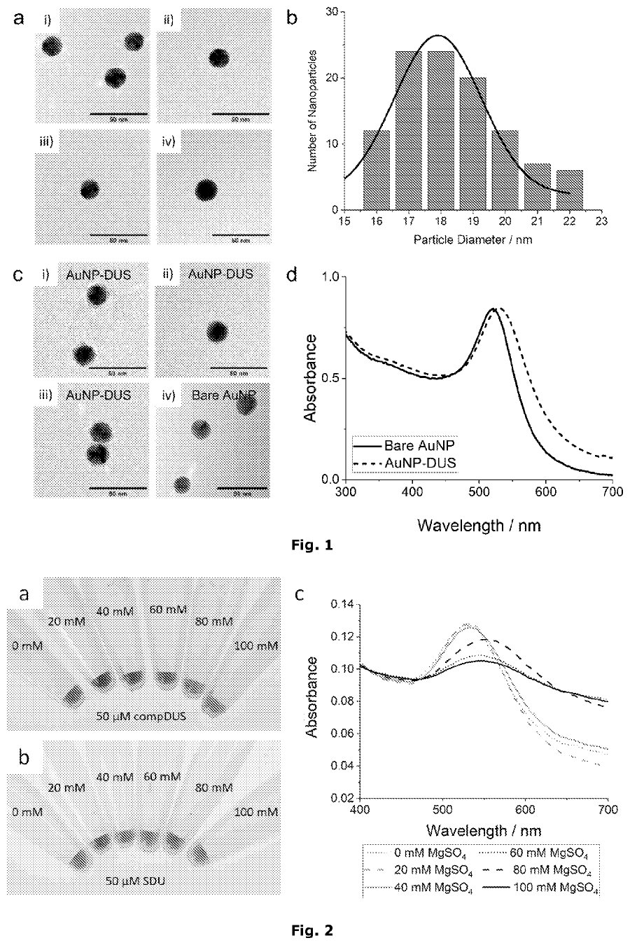

Spherical AuNPs with an average diameter of 18.3±1.7 nm were successfully produced as observed by TEM (FIGS. 1a and 1b). Dynamic light scattering measurements revealed an average hydrodynamic diameter of 28.9±0.4 nm and a large negative zeta potential (−29.0±4.9), accounted for by the electrostatically absorbed citrate groups. The AuNPs exhibited a maximum absorbance at 520 nm (see black curve in FIG. 1c) consistent with their predicted surface plasmon resonance (S. Link and M. A. El-Sayed, J. Phys. Chem. B, 1999, 103, 4212-4217).

Following surface-functionalisation of the AuNPs with a thiolated DUS-containing oligonucleotide, a 29 nm average increase in hydrodynamic diameter was observed, consistent with approximately two lengths of the solvated oligonucleotides. Conjugation of DUS DNA around the particle surface was confirmed by TEM imaging, where uranyl acetate-stained DNA was observed around the periphery of the nanoprobes but not for the bare AuNPs (see FIG. 1c). A small 9 nm increase in surface plasmon resonance was observed for the nanoprobes indicating the particles remain stable in solution following functionalisation with oligonucleotides. Cleavage of the gold-thiol bonds and subsequent quantification of the released DNA using a Quant-it OliGreen fluorescent reagent, revealed that the DUS functionalised nanoprobes contained approximately 145 oligonucleotides per AuNP.

### Example 2—Salt-Induced Aggregation for the Detection of Complimentary Oligonucleotides

Complementary annealing to a reverse complement DUS (compDUS) oligonucleotide conferred additional stability to the DUS-functionalised nanoprobes. At MgSO₄ concentrations of 40 mM and below, the particles treated with compDUS remained stable, as demonstrated by the red coloured suspensions (FIG. 2a) and constant SPR at 520 nm (red curves in FIG. 2c). In contrast, when treated with a non-complementary control oligonucleotide (SDU) the particles were only stable up to 20 mM but aggregated at ≥40 mM MgSO₄, as shown by a colour change from red to blue for all SDU-treated suspensions (FIG. 2b). At 60 mM MgSO₄ and above, all nanoconjugate samples aggregated irrespective of the presence of target DNA as shown by the blue suspensions in FIG. 2a. In agreement with the observed colour changes, the gold nanoparticle SPR red-shifted from 520 nm to longer wavelengths between 143-555 nm (blue curves in FIG. 2c). This result indicates that the additional stability conferred by target hybridisation was insufficient to withstand the charge screening introduced at higher Mg²⁺ concentrations. It was therefore determined that 40 mM MgSO₄ was the optimum concentration for identifying whether the nanoprobes have been treated with target DNA and was used henceforth.

### Example 3—Testing the Sequence Specificity of Aggregation Tests

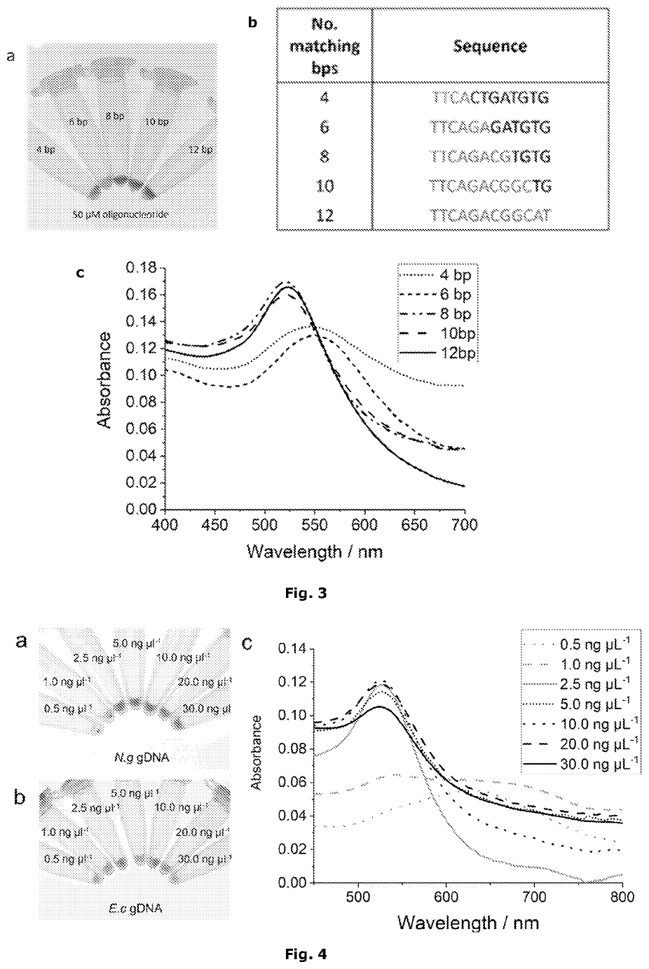

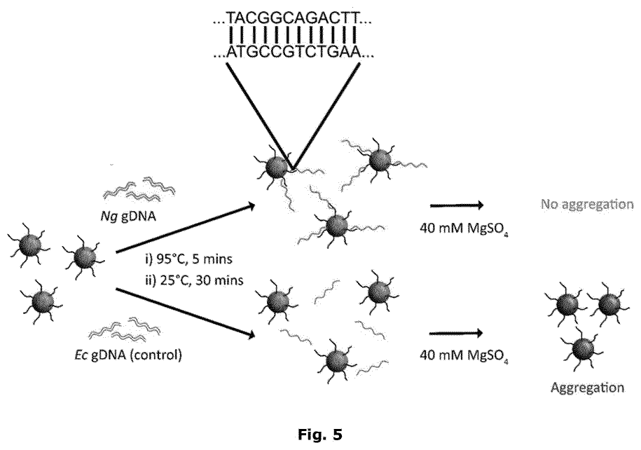

Oligonucleotides of ≤6 complementary bases (nucleotide sequences shown in FIG. 3b) provided no ultimate additional stabilisation to the nanoprobes as indicated by a red-to-blue colour change (FIG. 3a). In contrast, nanoprobes treated with oligonucleotides composed of ≥8 complimentary bases did not undergo any colour change (FIG. 3a), indicating these oligonucleotides are sufficient to bind genomic DNA and stabilise the nanoprobes against 40 mM MgSO₄. Supporting the observations, UV-vis spectroscopy revealed a red-shift in the nanoprobe SPR to 545-500 nm, when treated with oligonucleotides of ≤6 base matches (FIG. 3c). Together, these results suggest that at least 7 or 8 complimentary bases are required for hybridisation to occur between an oligonucleotide and the nanoprobes at room temperature with this particular system (i.e. the specific combination of the chosen gDNA sequence, the nanoprobe type and the magnesium concentrations used).

### Example 4—Salt-Induced Aggregation for the Detection of Gonococcal Versus Control Genomic DNA

To confirm specificity for Neisseria, the nanoprobe diagnostic was also tested with Stellar E. coli genomic DNA. E. coli was chosen since it is the predominant species found in the urethra, vagina and semen, all of which are common sites of gonococcal infection and thus, are commonly found in patient samples.

When incubated with N. gonorrhoeae (MS11) genomic DNA, the nanoprobes exhibited improved stability at 40 mM MgSO₄ compared to those incubated with E. coli (Stellar) genomic DNA. At gonococcal DNA concentrations of ≥2.5 ng μL⁻¹, the nanoprobes remained stable at 40 mM MgSO₄, as demonstrated by their red colour (FIG. 4a) and constant SPR at 520 nm (red curves in FIG. 4b). At DNA concentrations below this, particle aggregation occurred as indicated by a red-shift in the SPR to 630 nm (blue curves in FIG. 4b) and a red-to-blue colour change (FIG. 4a). In addition, some visible particle aggregates began to form 5 minutes after MgSO₄ addition, as shown by black precipitate in these two suspensions. In contrast, when treated with E. coli DNA, the nanoprobes aggregated at all concentrations as shown by distinct blue colour following MgSO₄ addition (FIG. 4b), confirming specificity for Neisseria. Again, the formation of some visible particle aggregates was also observed, demonstrating high nanoprobe instability in the presence of non-target genomic DNA. Together, these results indicate the DUS-functionalised nanoprobes can specifically detect the presence of gonococcal DNA within solutions with a detection limit of 2.5 ng μL⁻¹.

### Example 5—Complementarity Analysis in Stellar E. coli Genome

A CBI nucleotide BLAST of the DUS sequence against the Stellar E. coli genome, revealed a single complete DUS match with further partial matches. Since no particle stabilisation was observed with this DNA, this finding demonstrates that the relatively low frequency of complete and partial DUS matches within other genomes, compared to the 1521 complete matches in gonococcal DNA, is insufficient to cause a false-positive result.

1. A method for detecting target genomic DNA in a test sample, wherein the target genomic DNA comprises a DNA repeat sequence, said method comprising:
(a) providing a nanoprobe comprising a nanoparticle functionalised by a surface-mounted oligonucleotide, wherein the oligonucleotide comprises a sequence that is substantially complementary to a portion of the DNA repeat sequence,
(b) contacting the test sample with the nanoprobe,
(c) contacting the test sample with a nanoprobe aggregation agent, and
(d) assessing the amount of nanoprobe aggregation.

2. A method according to claim 1, wherein the target genomic DNA is genomic DNA of a target pathogen.

3. A method according to claim 2, wherein the target pathogen is a prokaryote, preferably a bacterium, more preferably a Gram-negative bacterium, yet more preferably Neisseria gonorrhoeae.

4. A method according to any preceding claim, wherein the DNA repeat sequence comprises at least 4, 6, 8, 10, 20, 40, 60, 80, 100, 200, 400, 600, 800, 1000, 1500, 2000, 2500, 5000, 10000, 20000, or 50000 repeats.

5. A method according to any preceding claim, wherein the DNA repeat sequence is a DNA uptake sequence, preferably a DNA uptake sequence in the genomic DNA of a bacterial species within the Neisseriaceae family or Pasteurellaceae family, more preferably a DNA uptake sequence in the genomic DNA of Neisseria gonorrhoeae or Haemophilus influenzae.

6. A method according to any preceding claim, wherein the oligonucleotide comprises a sequence that is complementary to a portion of the DNA repeat sequence.

7. A method according to any preceding claim, wherein the oligonucleotide comprises a sequence having at least 6 nucleotides that are complementary to a portion of the DNA repeat sequence.

8. A method according to any preceding claim, wherein the nanoparticle exhibits a different colour in a disperse state versus an aggregated state.

9. A method according to any preceding claim, wherein the nanoparticle is a gold nanoparticle.

10. A method according to any preceding claim, wherein the DNA aggregation agent is a charge screening agent, preferably a salt, more preferably a cation.

11. A method according to any preceding claim, wherein the nanoprobe aggregation agent is a magnesium salt.

12. A method according to claim 11, wherein the magnesium salt is added to the test sample in an amount that gives a magnesium salt concentration in the test sample of between 20 and 60 mM, preferably between 30 and 50 mM, more preferably about 40 mM.

13. A method according to any preceding claim, wherein the target genomic DNA is the DNA uptake sequence of Neisseria gonorrhoeae, and the nanoprobe comprises a gold nanoparticle functionalised by an oligonucleotide comprising a sequence that is complementary to at least 8 contiguous nucleotides of the DNA uptake sequence.

14. A method for detecting the presence of a target pathogen in a test sample, wherein the target pathogen comprises genomic DNA that comprises a DNA repeat sequence, said method comprising:
(a) providing a nanoprobe comprising a nanoparticle functionalised by a surface-mounted oligonucleotide, wherein the oligonucleotide comprises a sequence that is substantially complementary to a portion of the DNA repeat sequence,
(b) contacting the test sample with the nanoprobe,
(c) contacting the test sample with a nanoprobe aggregation agent, and
(d) assessing the amount of nanoprobe aggregation.

15. A method according to claim 14 wherein the test sample is a sample obtained from a subject, and wherein detection of a target pathogen is indicative of infection by the target pathogen.

16. A method of diagnosing infection with a target pathogen in a subject, using a test sample obtained from the subject, wherein the target pathogen comprises genomic DNA that comprises a DNA repeat sequence, said method comprising:
(a) providing a nanoprobe comprising a nanoparticle functionalised by a surface-mounted oligonucleotide, wherein the oligonucleotide comprises a sequence that is substantially complementary to a portion of the DNA repeat sequence,
(b) contacting the test sample with the nanoprobe,
(c) contacting the test sample with a nanoprobe aggregation agent, and
(d) assessing the amount of nanoprobe aggregation.

17. A nanoprobe comprising a nanoparticle functionalised by a surface-mounted oligonucleotide, wherein said oligonucleotide comprises a sequence that is substantially complementary to a portion of a DNA repeat sequence of target genomic DNA, wherein the target genomic DNA is genomic DNA of a target pathogen.

18. A nanoprobe according to claim 17, for use in the diagnosis of infection by the target pathogen.

19. A kit comprising:
(a) an analytical sample comprising a nanoprobe according to claim 17 or 18, and
(b) a nanoprobe aggregation agent.

20. A method for designing a nanoprobe, the method comprising:
(a) identifying a target pathogen comprising genomic DNA comprising a DNA repeat sequence,
(b) identifying the sequence of the DNA repeat sequence,
(c) designing an oligonucleotide that is substantially complementary to a portion of the DNA repeat sequence, and
(d) generating the nanoprobe by surface-mounting the oligonucleotide on a nanoparticle.
